(19) United States
(12) Patent Application Publication
(10) Pub. No.: US 2010/0075384 A1
(43) Pub. Date: Mar. 25, 2010

(54) Helicase-dependent amplification of circular nucleic acids

(75) Inventors: Huimin Kong, Wenham, MA (US); Yan Xu, Hamilton, MA (US)

Correspondence Address: HARRIET M. STRIMPEL, D. Phil., New England Biolabs, Inc., 240 COUNTY ROAD, IPSWICH, MA, 01938-2723, US

(73) Assignee: New England Biolabs, Inc., Ipswich, MA (US)

(21) Appl. No.: 10/594,095

(22) PCT Filed: Mar. 23, 2005
(86) PCT No.: PCT/US05/09829

§ 371 Date: Sep. 11, 2007

Related U.S. Application Data
(60) Provisional application No. 60/556,297, filed on Mar. 25, 2004.

(51) Int. Cl. C12P 19/34; C12N 9/10

(52) U.S. Cl. 435/91.2; 435/193

(57) ABSTRACT

A helicase-mediated amplification method for circular DNA templates and target DNA sequences within the templates is provided. The method combines a DNA polymerase and a helicase preparation to amplify a target sequence as well as the entire circular DNA template.

### FIELD OF INVENTION

Embodiments of this invention relate to methods for amplifying circular nucleic acids using a DNA helicase and two sequence-specific primers.

### BACKGROUND

DNA amplification has become a basic technique in biological studies. The most widely used amplification method is the polymerase chain reaction (PCR), which uses a thermostable DNA polymerase to amplify a specific sequence defined by two primers (for example, U.S. Pat. Nos. 4,683,195, 4,683,202 and 4,800,159). Denaturation of duplex DNA in PCR is achieved by temperature cycling in a thermocycler.

The requirement for a thermocycler in PCR restricts its portable use. Alternative isothermal techniques that do not utilize thermocycling have been developed. Helicases have been used for isothermal nucleic acid amplification (US publication No. US-2004-0058378-A1). Another approach requires extending nicked double stranded DNA using a nuclease-deficient DNA polymerase. (See, for example, U.S. Pat. Nos. 5,455,166 and 5,470,723).

An isothermal amplification procedure for circular DNA called Rolling Circle Amplification (RCA) has been developed. When one primer is used in RCA, a linear RCA amplification produces a continuous sequence of tandem copies of the circle DNA templates due to the strong strand-displacement activity of a DNA polymerase [Fire, A. and Si-Qun Xu, Proc. Natl. Acad Sci. USA 92:4641-4645 (1995); Lui, et al., J. Am. Chem. Soc. 118:1587-1594 (1996)]. Two primers can be used with a small (less than 150 bp) circularized padlock DNA probe to produce hyperbranched amplification of the entire small circle [Lizardi, et al., Nature Genetics 19:225-232 (1998), Zhang, et al., Gene 211:277-285 (1998)]. Multiply-primed RCA utilizes random hexamers as primers using Phi29 DNA polymerase [Dean et al., Genome Res. 11:1095-1099 (2001)]. The amplification product is difficult to manipulate because it is highly branched and viscous.

Development of a technology that is able to amplify specific regions in a circular DNA as well as the entire circular DNA (cDNA) would be a useful DNA production and diagnostics technique. Also useful would be a single convenient step to screen or identify plasmids.

### SUMMARY OF THE INVENTION

In an embodiment of the invention, a method of circular-Helicase-Dependent Amplification (cHDA) is provided for amplifying nucleic acids from a cDNA template. This system combines a DNA polymerase and a helicase preparation to amplify a target sequence as well as the entire circular DNA template containing the target sequence.

In an embodiment of the invention, a method is provided for amplifying a target DNA sequence in a cDNA. A first sequence-specific DNA primer that hybridizes to one strand of the cDNA, a second sequence-specific DNA primer that is substantially identical to a portion of the same strand of the cDNA, a helicase preparation, a DNA polymerase and dNTPs are added to the cDNA template. Primer extension products are synthesized from the primers annealed to the cDNA. The product of the amplification is a plurality of copies of the target DNA sequence as defined by the first and second primers and a plurality of copies of a concatamer derived from the cDNA, including the target DNA sequence.

Additional details about the primer extension products include: replicating the cDNA under conditions whereby the first primer is extended around the circle repeatedly to generate a single strand linear concatemer, and whereby the second primer hybridizes to multiple sites on a complementary single-stranded DNA which generates extension products for continuing synthesis of the target DNA sequence and the cDNA.

In another embodiment of the invention, a method for amplifying a target DNA sequence in a cDNA is provided that includes: (a) annealing a first sequence-specific oligonucleotide primer to a first DNA sequence adjacent to or within a target DNA sequence in a cDNA in the presence of a polymerase and a helicase preparation to synthesize by primer extension a displaced single-stranded DNA containing a plurality of copies of the target DNA sequence; (b) annealing a second sequence-specific oligonucleotide primer to the displaced strand of DNA at specific sites for synthesizing by primer extension a plurality of complementary single-stranded DNA concatamers, each concatamer including the target DNA sequence and each concatemer forming a substrate for multiple rounds of displacement synthesis from the first or second primer; and (c) amplifying the target DNA sequence.

The target DNA sequence can include all or part of the cDNA where the part of the cDNA that is the target sequence is defined by the first and second primers. The first and second primers may recognize sequences that overlap or are distinct in the cDNA. The cDNA can be double- or single-stranded or part double-stranded and part single-stranded with a size in the range of 50 nucleotides to 500 kb. The cDNA may be a plasmid. It may be extrachromosal for detecting pathogens. Moreover, the cDNA may be purified or may be a crude preparation obtained from lysis of a culture of cells containing cDNAs. The cDNA may be a plasmid, mitochondrial DNA, chloroplast DNA, closed padlock probes or circularized linear DNA.

When the DNA is double-stranded in a sample or in a culture, it may be converted into complete or partial single-stranded form by means of heat-denaturation, chemical treatment, or helicase-unwinding to provide single stranded templates for amplification. However, during amplification, unwinding of the duplex DNA is achieved by means of a helicase.

In an embodiment of the method, the DNA polymerase is selected from a T7 bacteriophage, a T7-like polymerase or an exonuclease deficient variant thereof.

An embodiment of the invention features a method for selectively amplifying a target DNA sequence and/or the entire sequence of a cDNA template, that includes: (a) adding to the DNA, a helicase preparation containing one or more helicases, a nucleotide triphosphate (NTP) or deoxynucleotide triphosphate (dNTP), a buffer, and, optionally, one or more single-stranded DNA binding proteins and/or one or more accessory proteins; (b) adding a target nucleic acid in varying concentrations or copy number, oligonucleotide primers and a DNA polymerase to the helicase preparation; (c) incubating the mixture at a temperature between approximately 15° C. and 75° C. for up to several days; and (d) analyzing the amplified DNA by, for example, gel electrophoresis or capillary electrophoresis to determine whether amplification has occurred. The composition of the reaction mixture, conditions of the reaction and concentration of the reactants can be varied within certain ranges provided herein to identify the optimum conditions for helicase-mediated cDNA amplification. An example of a buffer that is suitable for use in amplification is Tris-acetate or Tris-HCl at a pH in the range of approximately pH 5.5-10.0, and a concentration of NaCl or KCl in a concentration range of 0-200 mM.

In an embodiment of the methods described herein, the helicase preparation may include a single helicase or a plurality of helicases. The helicase or helicases in the preparation may be selected from the class of 5′ to 3′ helicases and/or the class of 3′ to 5′ helicases. The one or more helicases may include a hexameric helicase or a monomeric or dimeric helicase. For example, the helicase may be a T7 bacteriophage or T7-like bacteriophage helicase. The helicase preparation may also include a single-strand binding protein from a T7 bacteriophage or T7-like bacteriophage, and cofactors such as nucleotides.

In an embodiment of the invention, amplification is isothermal and may be accomplished in the range of approximately 15° C.-75° C., preferably at ambient room temperature (25° C.).

In an embodiment of the invention an amplification kit is provided containing a helicase preparation containing one or more helicases, a DNA polymerase and instructions for performing helicase-dependent amplification of circular nucleic acids. More particularly, the kit may contain one or more of the following: T7 gene 4B helicase, T7 gene 2.5 SSB protein, dTTP or ATP and T7 Sequenase, one or more cofactors including an accessory protein, a set of four deoxynucleotides and optionally a reaction buffer.

In an embodiment of the invention, the helicase preparation includes an NTP or dNTP, for example, adenosine triphosphate (ATP), deoxythymidine triphosphate (dTTP) or deoxyadenosine triphosphate (dATP). A suitable concentration for the energy source is in the range of approximately 0.1-100 mM.

The amplification products can be used directly for sequencing, enzyme digestion, cloning and other applications.

### BRIEF DESCRIPTION OF THE DRAWINGS

FIG. 1 is a schematic diagram of circular helicase-dependent amplification to produce both target DNA and template DNA corresponding to the entire linearized cDNA

FIG. 1A shows some of the different forms of cDNA that may be used as a substrate for cHDA amplification. (1) is a double-stranded cDNA in duplex form with a positive strand shown as a solid circle and a −strand shown in a dashed circle; (2) is a double-stranded duplex cDNA with single-stranded region to which complementary primers (shown as lines with arrows) can bind; (3) is a double-stranded cDNA in denatured form providing access to primer-binding; and (4) is a single-stranded cDNA.

FIG. 1B shows an antisense primer (solid lines with arrow head) annealing to the template. Primer extension produces a concatemer of the template. Multiple sense primers (dotted lines with arrow head) anneal to the concatemer and are extended by the DNA polymerase. As the polymerization reaches the downstream primer extension product, the helicase/DNA polymerase complex displaces the non-template strand. Multiple rounds of displacement and polymerization produce a specific target DNA defined by two primers and multimers of the DNA template.

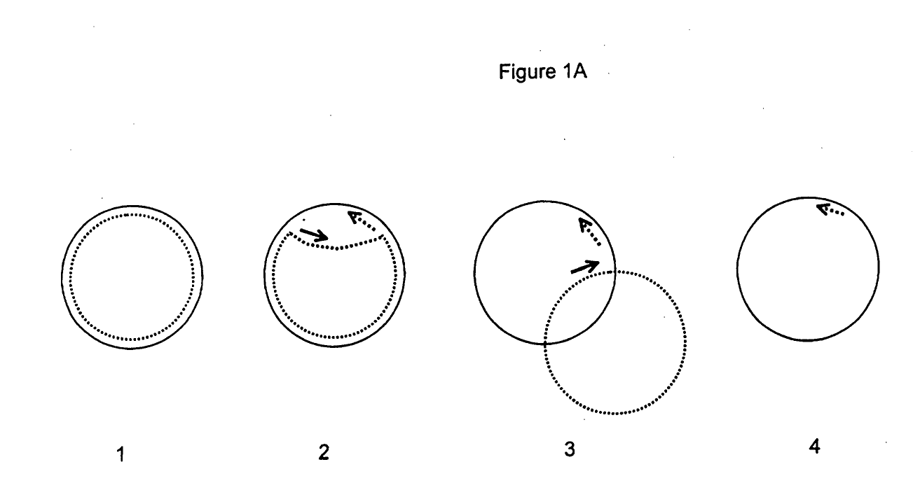

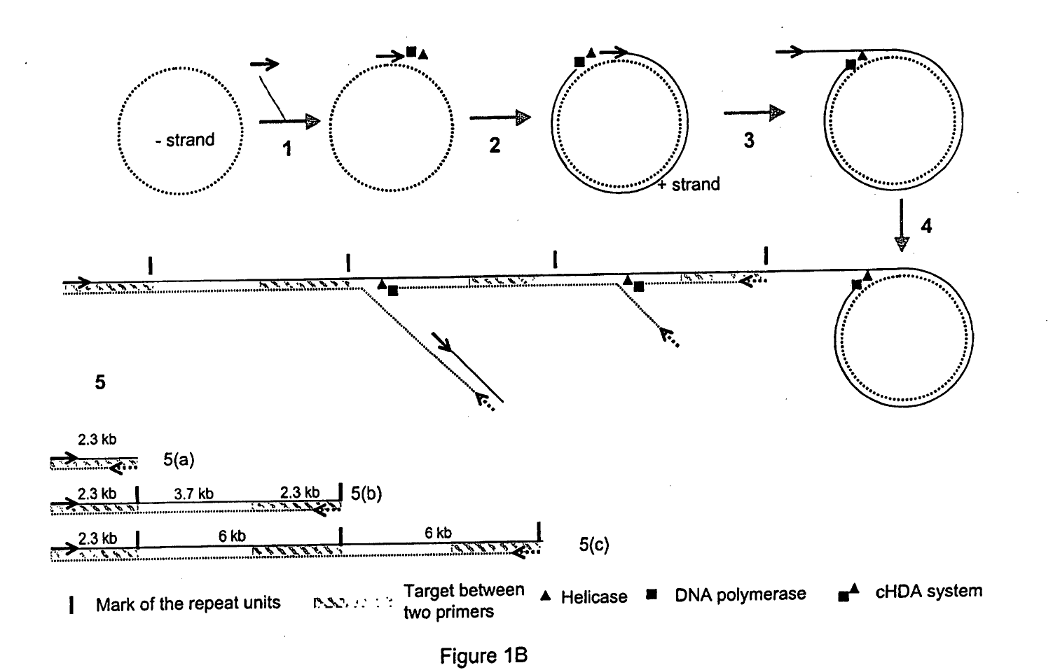

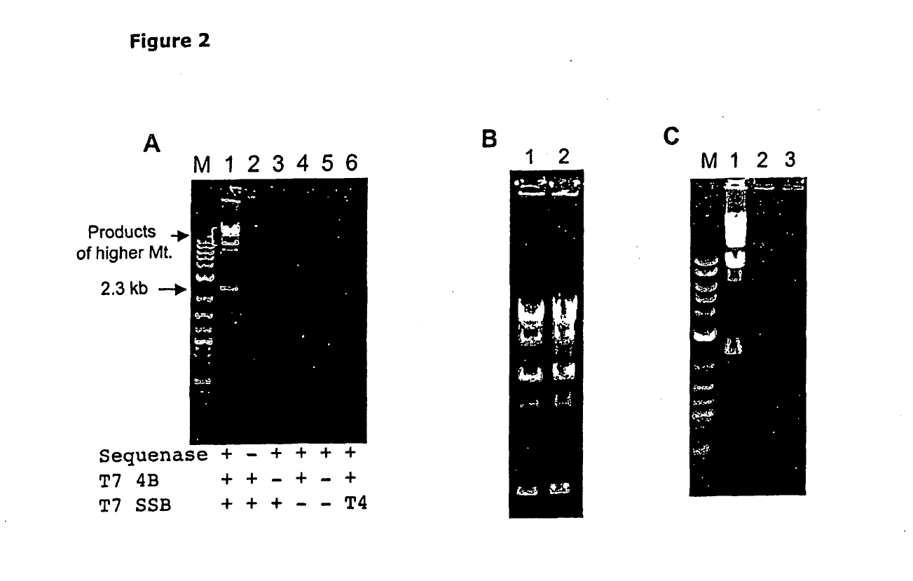

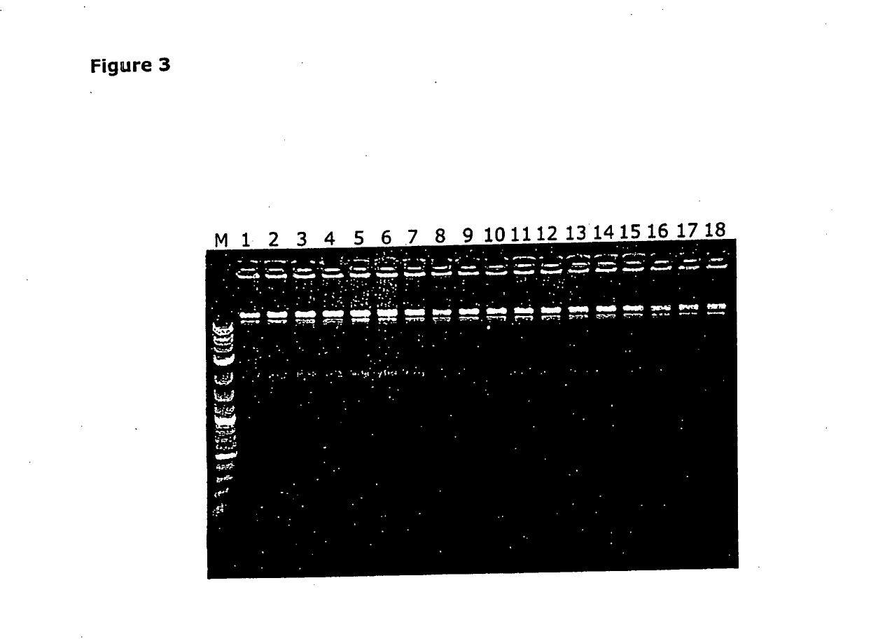

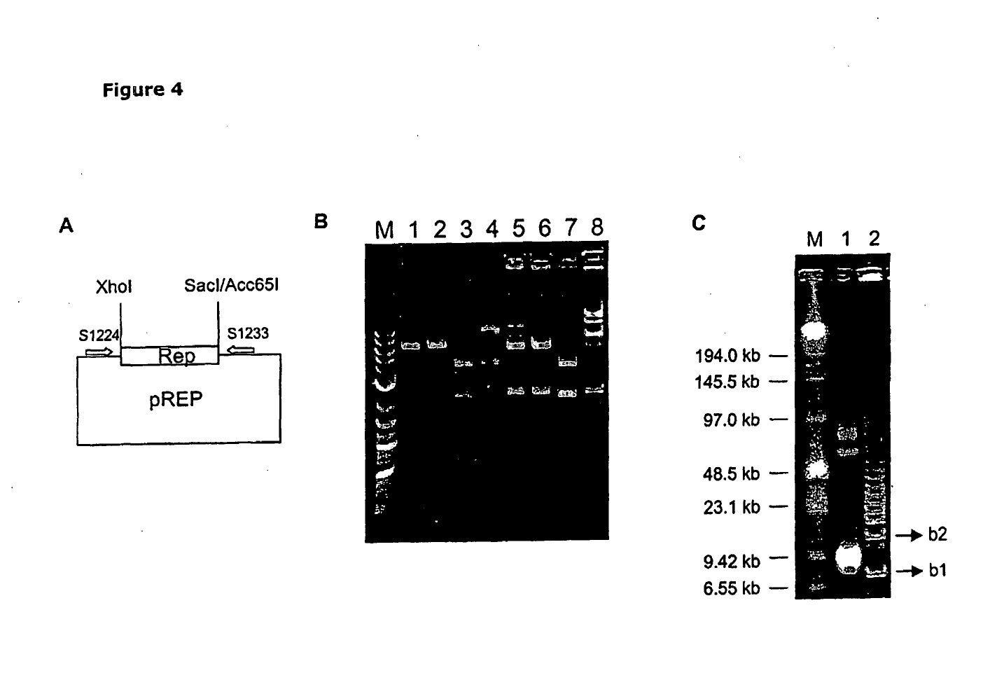

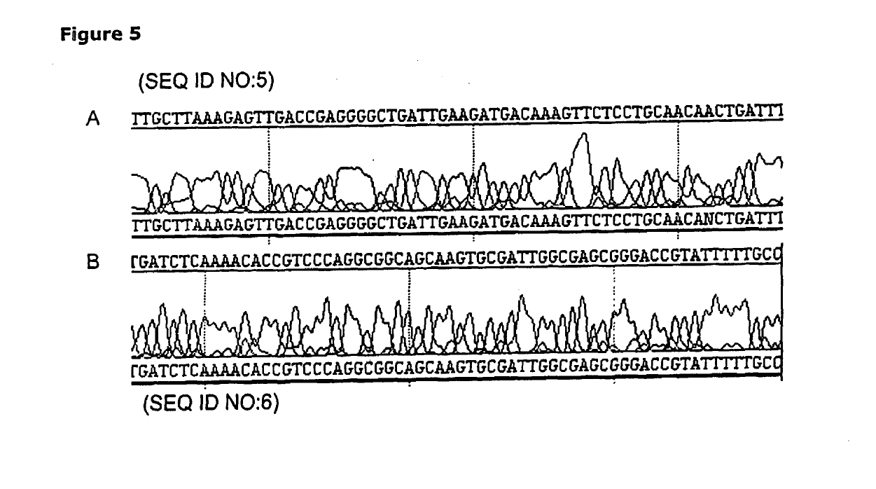

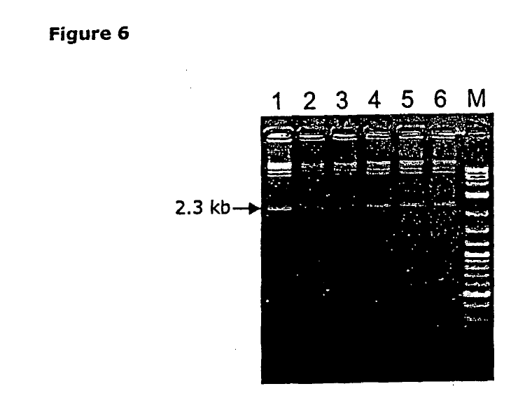

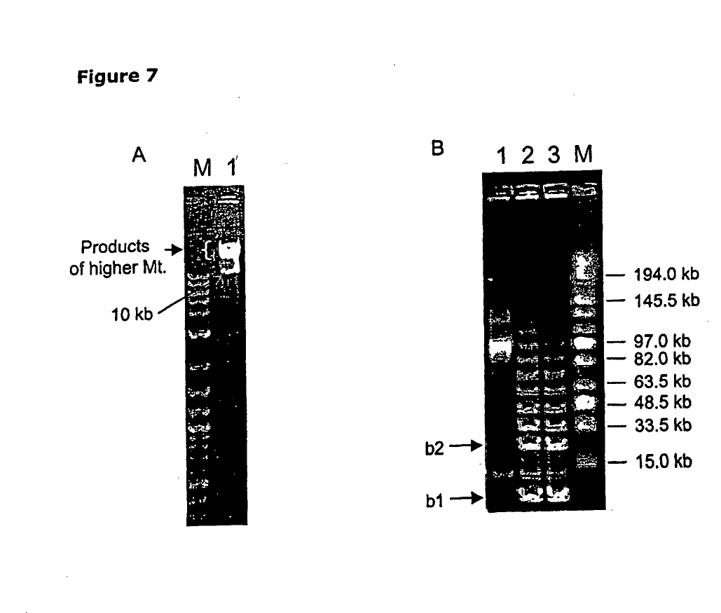

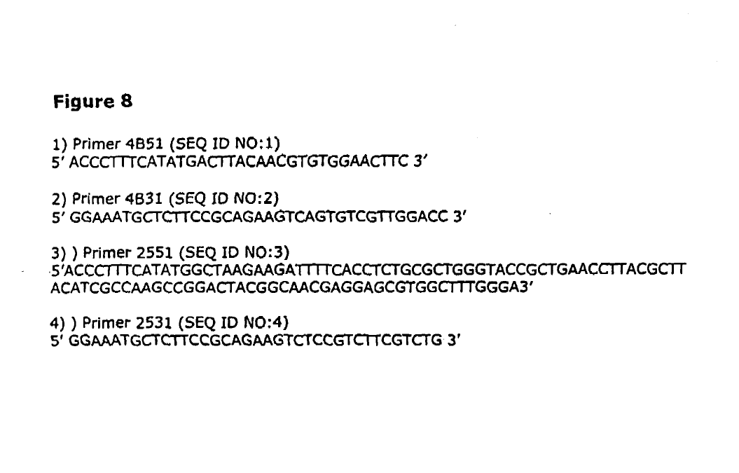

FIG. 1B show a cHDA reaction starting on a − strand of a cDNA.
Step 1: First primer annealing to the − strand (shown in dashed circle);
Step 2: First primer extension;
Step 3: Unwinding by a helicase displaces the + strand (shown in solid line);
Step 4: Continued synthesis of the + strand by primer extension of the first primer producing a linear displaced + strand. The second primer binds to the displaced strand at a specific sequence at multiple positions and initiates multiple displacement synthesis of both the defined target DNA and the entire DNA;
Step 5: The amplified product is a 2.3 kb target fragment (5a), a 2.3 kb target sequence and an entire 6 kb cDNA template (5b), and the 2.3 kb target plus concatamers of the cDNA template (5c). The 6 Kb cDNA consists a 2.3-kb target region and a 3.7-kb plasmid backbone.

Helicase ▾ DNA polymerase ▪ cHDA system ▪▾ repeat units marked by |

FIG. 2 shows cHDA using plasmids as templates

FIG. 2A shows the results of cHDA amplification of plasmid pREP in the presence of essential components of cHDA using 1% agarose gel electrophoresis. The lanes are as follows:
Lane 1: a complete reaction mix;
Lane 2: a complete reaction mix minus Sequenase;
Lane 3: a complete reaction mix minus T7 gp4B helicase;
Lane 4: a complete reaction mix minus T7 gp2.5 SSB protein;
Lane 5: a complete reaction mix minus both T7 gp4B helicase and gp2.5 SSB protein; and
Lane 6: the result of substitution of T7 gp 2.5 SSB with the gene 32 SSB protein from the T4 bacteriophage. M: 2-log DNA ladder (New England Biolabs, Inc., Beverly, Mass.).

FIG. 2B shows a comparison of cHDA reactions with or without a prior heat-denaturation step by gel electrophoresis. cHDA amplification was performed on plasmid pREP. The lanes are as follows: lane 1, one-step cHDA without heat-denaturation and, lane 2, two-step cHDA with a prior heat-denaturation step.

FIG. 2C shows that exponential cHDA amplification requires two primers using 1% agarose gel electrophoresis. The lanes are as follows:
Lane 1: both the specific 2.3 kb fragment and larger fragments were amplified in the presence of two primers: 20 pmole each of oligonucleotide primers S1224 and S1233 (New England Biolabs, Inc., Beverly, Mass.);
Lane 2, 20 pmole of only primer S1224 (no amplification product); and
Lane 3, 20 pmole of only primer S1233 (no amplification product); lane M, 2-log DNA ladder (New England Biolabs, Inc., Beverly, Mass.).

FIG. 3 shows optimization of cHDA buffer conditions by 1% gel electrophoresis. Compared to the reaction containing no additives (lane 1), the presence of Triton X-100, ZnCl₂ or potassium glutamate improved amplification yield.
Lane 1: A positive control was performed in which no additional reagents were added to the cHDA reaction;
Lanes 2-18 include additional reagents as follows:
Lane 2, 0.1% Triton X-100 supplement;
Lane 3, 0.075% Triton X-100 supplement;
Lane 4, 0.05% Triton X-100 supplement;
Lane 5, 0.025% Triton X-100 supplement;
Lane 6, 0.0125% Triton X-100 supplement;
Lane 7, 50 mM ZnCl₂ supplement;
Lane 8, 25 mM ZnCl₂ supplement;
Lane 9, 12.5 mM ZnCl₂ supplement;
Lane 10, 6.25 mM ZnCl₂ supplement;
Lane 11, 3.13 mM ZnCl₂ supplement;
Lane 12, 1.56 mM ZnCl₂ supplement;
Lane 13, 1 mM potassium glutamate supplement;
Lane 14, 0.8 mM potassium glutamate supplement;
Lane 15, 0.4 mM potassium glutamate supplement;
Lane 16, 0.2 mM potassium glutamate supplement;
Lane 17, 0.1 mM potassium glutamate supplement;
Lane 18, 0.05 mM potassium glutamate supplement.

FIG. 4 is an analysis of cHDA products.

FIG. 4A is a diagram of plasmid pREP. The relative positions of primers and restriction enzyme sites are indicated.

FIG. 4B shows restriction enzyme digestion of cHDA products. When the purified pREP plasmid was digested with either Acc65I or SacI, pREP was linearized and produced a 6-kb fragment (lanes 1 and 2). Digestion of pREP with XhoI and SacI yielded two bands, a 2.2 and 3.8-kb fragments (lane 3). Similarly, digestion of the amplification pREP product with either Acc65I or SacI produced the specific 2.3 kb fragment, in addition to the linearized 6 kb plasmid (lanes 5 and 6). When the amplification product was digested with XhoI and SacI, 3.8 and 2.2 kb fragments were produced. The 2.3 and 6 kb bands were visible in a single SacI digestion, which was further cleaved into 2.2 kb and 3.8 kb by XhoI (lane 7). The lanes are as follows: lane 1, Acc65I digestion of pREP; lane 2, SacI digestion of pREP; lane 3, SacI and XhoI digestion of pREP; lane 4, pREP plasmid undigested; lane 5, Acc65I digestion of the cHDA product; lane 6, SacI digestion of the cHDA product; lane 7, SacI and XhoI digestion of cHDA product; lane 8, the undigested cHDA reaction product; and the 2-log DNA ladder (New England Biolabs, Inc., Beverly, Mass.) is indicated as ‘M.’

FIG. 4C shows pulse-field gel (PFG) electrophoresis of cHDA products. Lane 1 shows the original plasmid and lane 2 shows the amplification product. The smallest band, b1, is approximately 8.3 kb in size and matches the predicted size of the pREP plasmid (6 kb) plus the insert (2.3 kb). The next band, b2, is approximately 14.3 kb, which also corresponds to the predicted size (2×6 kb+2.3 kb). The lanes are as follows: M, low range PFG DNA size marker (New England Biolabs, Inc., Beverly, Mass.); lane 1, template plasmid pREP as the starting material; lane 2, the cHDA product amplified from 10 ng of plasmid pREP.

FIG. 5 shows direct sequencing reactions using cHDA products.

The sequencing reactions produced strong peaks with low background, indicating that the cHDA products can be used as sequencing templates without an initial purification step. Results of the sequencing are shown: panel A, a region of the sequencing trace data using primer S1224 (SEQ ID NO:5); panel B, a region of the sequencing trace data using a Rep specific primer (SEQ ID NO:6).

FIG. 6 shows the performing of cHDA from a bacterial colony containing plasmids.

A purified plasmid DNA reaction (plasmid pREP) was performed (lane 1). Parallel cHDA reactions were performed using crude plasmid DNA directly from bacterial colonies: lane 2, 1 ml; lane 3, 2 ml; lane 4, 3 ml; lane 5, 4 ml; and, lane 6, 5 ml. The 2-log DNA ladder (New England Biolabs, Inc., Beverly, Mass.) is indicated as ‘M.’

FIG. 7 shows amplification of a 10-kb fragment by cHDA.

FIG. 7A shows amplification of a 10 kb fragment from pTopo10k. Lane 1 contains the pTopo10k cHDA reaction and, lane M, is the 2-log DNA ladder (New England Biolabs, Inc., Beverly, Mass.).

FIG. 7B shows pulse-field gel electrophoresis of pTopo10k cHDA products. The smallest band, b1, is approximately 10 kb, which is the specific band. The next band, b2, is approximately 24 kb and is consistent with the predicted size (14 kb of pTopo10k+10 kb insert). The lanes are as follows: lane 1, plasmid pTopo10k; lanes 2 and 3, the cHDA amplification product from pTopo10k; and, lane M, Mid-range PFG marker (New England Biolabs, Inc., Beverly, Mass.).

FIG. 8 shows sequence listings for primers 4B51, 4B31, 2551 and 2531 (SEQ ID NOS:1-4, respectively).

### DETAILED DESCRIPTION OF EMBODIMENTS

Helicase-dependent amplification (HDA) has been adapted for use in amplifying cDNA and additionally defined sequences (target sequences) within a cDNA in a procedure referred to here as cHDA. cHDA combines a DNA polymerase and primers with a helicase preparation to simultaneously amplify a specific sequence within a cDNA template such as a plasmid as well as the entire cDNA molecule. This approach provides a rapid screening method for determining the presence of defined sequences in a cDNA such as a plasmid.

While not wishing to be limited by theory, it is believed that during amplification in the presence of a helicase preparation, a sense primer and an antisense primer, the annealed sense primer is continuously extended and displaced by the cHDA system to form concatemers to provide multiple annealing sites for the reverse primer. Multiple rounds of strand-displacement synthesis by the cHDA system produce specific target DNA fragments defined by the two primers and multimers of the cDNA.

Certain terms employed in the specification, examples and appended claims are collected here.

The term “DNA” refers to double-stranded or single-stranded molecules. Linear DNA may be circularized. cDNA can be supercoiled or nicked. cDNA may occur naturally in the form of plasmids from bacteria, viral genomes, mitochondrial DNA or chloroplast DNA or any other DNA or may be produced by in vitro ligation or chemical synthesis. The DNA may be isolated from a variety of sources, including the environment, food, agriculture, fermentations, biological fluids, biological tissue samples or cells. Circular nucleic acids for use in cHDA may be obtained from sources or preparations that differ in extent of purity and which may contain non-target DNA other than sequences in the target DNA. There is no upper limit on the size of target DNA or cDNA template although in embodiments of the invention, a size range of 50 bp-500 kb is provided. cHDA can be used to amplify DNA that contain modified nucleotides. “Modification” refers to individual nucleotides within the nucleic acid that are chemically altered (for example, by methylation). Modifications may also arise naturally or by in vitro synthesis.

Sequence-specific oligonucleotide primers initiate polymerase-dependent replication of the target DNA. In a preferred embodiment, the primers are defined by the specific sequence to which they anneal. For amplification, one or more primer pairs are used where each primer in the pair prime DNA replication on opposite strands of a DNA duplex. The primer pairs may be orientated with respect to each other either “head to head” meaning 3′ to 3′ or “tail to tail” meaning 5′ to 5′. The primers may be overlapping on opposite strands or may be separated by the distance of the target DNA sequence. The primer is commonly depicted as having an arrow to denote orientation from 5′ to 3′ where initiation of primer extension occurs at the 3′ end.

To initiate amplification, only one primer is required to anneal to a single-stranded cDNA (see FIG. 1B). The amplification of DNA between two primers occurs from complementary DNA synthesized from the displaced strand, which in turn is a product of primer extension of the cDNA.

To simplify the detection of amplification products, the primers may be subject to modification, such as fluorescent- or chemiluminescent-labeling and biotinylation. Other labeling methods, including radioactive isotopes, chromophores and biotin or hapten ligands, allow detection through the specific interaction with labeled molecules, like streptavidin and antibodies.

A “helicase preparation” refers to a mixture including one or more helicases and at least one additional component. A helicase preparation may include an accessory protein, a single-stranded DNA binding (SSB) protein, small molecules, chemical reagents and/or buffer.

Helicases are enzymes that catalyze the unwinding of nucleic acid duplexes in cells during DNA replication and use energy provided by cofactors. (Kornberg, DNA Replication, W.H. Freeman and Company (2^{nd} edit. (1992), especially Chapter 11), NY, N.Y. Any helicase that translocates along DNA or RNA in a 5′ to 3′ direction or in the opposite 3′ to 5′ direction may be used in present embodiments of the invention.

Helicases are found in almost all organisms and can be obtained from prokaryotes, viruses, archaea, and eukaryotes. Alternatively, helicases can be recombinant forms of naturally occurring enzymes, analogues or derivatives with a specified activity. Examples of naturally occurring DNA helicases (see Kornberg and Baker; DNA Replication, W.H. Freeman and Company, 2^{nd} edit. 1992) include the E. coli helicase I, II, III, & IV, Rep, DnaB, PriA, and PcrA; the bacteriophage T4 gp41 and Dda helicases; bacteriophage T7 gp4 helicases; SV40 Large T antigen; Rho helicase; and yeast RAD. Examples of helicases for use in present embodiments may also be found at the following web address: http://blocks.fhcrc.org (Get Blocks by Keyword: helicase).

In an embodiment of the invention, a T7 helicase is used. In phage T7, gene 4 encodes two polypeptides that have helicase activity: a full-length 63-kDa gene 4A protein containing both primase and helicase functions, and a N-terminal truncated 56-kDa gene 4B protein containing 5′-3′ helicase activity with a high rate of processivity [Lechner and Richardson, J. Biol. Chem. 258:11185-11196 (1983); Kornberg and Baker, DNA Replication, W.H. Freeman and Company, 2^{nd} edit. (1992), NY, N.Y.; Kim et al., J. Mol. Biol. 21:807-819 (2002)]. The amino-terminal truncated version of the gene 4 protein (T7 4B helicase) which contains the DNA helicase activity is suitable for use in cHDA. The T7 4A helicase that has both the primase and helicase activities or a mixture of both 4A:4B helicases in varying 4A:4B ratios such as 1:1, 2:1 or 1:2 may also be used for cHDA.

The unwinding activity of a helicase during DNA amplification may use a cofactor that provides an energy source, such as an NTP or dNTP. Examples include: ATP (adenosine triphosphate) as a cofactor at a concentration in the range of 0.1-100 mM (for example 3 mM) and dTTP (deoxythymidine triphosphate) as a cofactor for T7 gp4B helicase. The cofactors may be used, for example, at a concentration in the range of 0.1-100 mM or in a range of 1-20 mM.

The activity of helicases may be further enhanced with additional small molecule reagents such as magnesium (or other divalent cations).

Accessory proteins are optionally included in the helicase preparation. The accessory proteins may include any protein capable of stimulating helicase activity. For example, E. coli MutL protein is an accessory protein (Yamaguchi et al. J. Biol. Chem. 273:9197-9201 (1998); Mechanic et al., J. Biol. Chem. 275:38337-38346 (2000)) for enhancing UvrD helicase melting activity (FIG. 1) in a UvrD helicase preparation. Bacteriophage T4 gene 32 coded protein (gp32) is an accessory protein which stimulates melting of DNA duplexes by trapping the separated ssDNA strands. In embodiments of the method, one or more accessory proteins may be included in the helicase preparation to enhance cHDA. For example, T7 SSB protein enhances unwinding activity of T7 helicase. Another accessory protein is Thioredoxin.

A helicase preparation may include a buffer that is suitable for enhancing amplification of DNA. An example of such a buffer is a salt buffer such as Tris-Cl or Tris-Acetate. Magnesium and DTT may be used in the helicase preparation.

For amplification of cDNA, the helicase preparation is combined with a polymerase and dNTPs in a “cHDA system”. The cHDA system refers to the reagents used to amplify the template and target DNAs. In embodiments of the invention, cHDA systems may contain a helicase, an SSB protein and additionally a polymerase from any T7-like phage such as T7, T3, phiI, phiII, H, W31, gh-1, Y, All22, or SP6 [Studier, Virology 95:70-84 (1979)]. In an embodiment of the invention, 200 to 2000 ng of the helicase may be included in a cHDA system.

The DNA polymerase in the cHDA system is preferably a processive polymerase, which lacks 5′ to 3′ exonuclease activity and possesses strand-displacement activity. The DNA polymerase may contain modifications or mutations that minimize its exonuclease activity or enhance its processivity, polymerization speed, or strand-displacement activity. A plurality of DNA polymerases and/or accessory proteins may be included in the cHDA system to enhance amplification rates, fidelity or size of amplicon.

In an embodiment of the invention, the cHDA system includes a T7 DNA polymerase. T7 polymerase is composed of two polypeptides: the T7 gene 5 protein and E. coli thioredoxin. Together they form a tightly associated complex and the thioredoxin domain confers high processivity to the DNA polymerase. The T7 DNA polymerase can polymerize more than 70 kb in one binding event at a speed of approximately 300 nt/sec (Kornberg and Baker, DNA Replication, W.H. Freeman and Company, 2^{nd} edit. 1992, NY, N.Y.). The wild-type T7 polymerase has a high fidelity rate during DNA replication with an error rate of 1.5×10⁵ [Korpela et al., Nuc. Acid. Res. 19:4967-4973 (1991)]. The 3′ to 5′ exonuclease activity of T7 DNA polymerase encoded by the gene 5 protein can be selectively inactivated by reactive oxygen species or substitutions of key amino acid residues in an exonuclease active site [Tabor and Richardson, J. Biol. Chem. 264:6647-6658 (1987)]. This modified exonuclease deficient form of the T7 DNA polymerase is commercially available as Sequenase™ version 2.0 [Tabor and Richardson, J. Biol. Chem. 264:6647-6658 (1987); USB Corporation, Cleveland, Ohio] and has strand-displacement activity while lacking 3′-5′ exonuclease activity.

Although the modified T7 DNA polymerase has lower or no 3′-5′ exonuclease activity and, therefore, lower fidelity during DNA synthesis, it can initiate strand-displacement synthesis at a nick, unlike the native T7 DNA polymerase. For cHDA reactions, 1-4 units of Sequenase™ may be used to support amplification.

In an embodiment of the cHDA system, the accessory protein, T7 gene 2.5, is included as a SSB protein. This protein has high homologous annealing capability [Yu and Masker, J. Bacteriol. 183:1862-1869 (2001)]. The T7 SSB protein interacts with both T7 DNA polymerase and T7 gene 4 proteins to stimulate both polymerase and helicase activity [Kim et al., J. Biol. Chem. 267:15032-15040 (1992); Notarnicola et al., J Biol. Chem. 272:18425-33 (1997)]. E. coli SSB protein, T7 gene 32 SSB protein and/or mutant alleles of gene 2.5 may be used instead of T7 gene 2.5 protein in cHDA reaction.

In the Examples, a cHDA system is described which is derived from T7. A T7 gp4B helicase and a T7gp2.5 SSB are combined with a T7 sequenase (polymerase). The unwinding activity of T7 gp4B helicase or a modified form thereof utilizes dTTP as an energy source and its activity is enhanced with T7 gp2.5 and/or the zinc-binding domains of the T7 gene 4 protein where T7gp 2.5 is capable of binding approximately 7 nucleotides per monomer [Kim et al., J. Biol. Chem. 267:15032-15040 (1992)].

The cHDA system may includes two or more oligonucleotide primers, each hybridizing to the borders of the specified target sequence or to a specific site within a cDNA if the whole molecule only is to be amplified.

A reaction buffer for use in cHDA may for example, contain a mixture of salts including Tris-Cl or Tris-Acetate, a magnesium source such as magnesium acetate or magnesium chloride and DTT in varying concentrations. The pH of the reaction buffer may range from 5-10 pH with an optimal pH of 7. Other chemical reagents, such as denaturation reagents, including urea and dimethyl-sulfoxide, may be added to the cHDA reaction to partially denature or destabilize the duplex DNA. Molecular crowding reagents, such as polyethylene glycol (PEG), may be included to improve the efficiency and yield of the amplification. Protein stabilizing reagents, such as trehalose, may be included to improve amplification. In addition, amplification may be increased by supplementing reactions with 0.001-0.1% Triton X-100; 1-50 mM ZnCl₂; and 0.05-1 mM potassium glutamate. An example of how the buffer can be optimized for a cHDA system as applied to the T7 cHDA system is shown in FIG. 3.

The following additional factors may be optionally added to the amplification mix: NTP or dNTP regeneration system or topoisomerase (Kornberg and Baker, DNA Replication, W.H. Freeman and Company, 2^{nd} edit. 1992, NY, N.Y.). Topoisomerase may be included to release the tension present in duplex DNA.

Temperatures for performing cHDA may range from 15° C. to 75° C. A preferred temperature for performing cHDA in field studies is ambient room temperature (about 25° C.). An initial denaturation step of the input DNA by temperature, chemical and/or helicase may not be necessary (FIG. 2). As such, reactions may be performed isothermally where “isothermal amplification” refers to amplification that occurs at a single temperature. This does not include the single brief time period (less than 15 minutes) at the initiation of isothermal amplification, which may be conducted at the same temperature as the amplification procedure or at a higher temperature. The ability to perform cHDA reactions isothermally means that instruments like thermocyclers are not necessary to perform amplification.

Advantages of c-HDA Over Other Amplification Systems

Circular HDA has improved characteristics over amplification procedures described for cDNA in the prior art, including:

(a) The target sequence is preferentially amplified over the remaining plasmid sequence, providing extra material for downstream sequencing and analysis.

(b) The cHDA method can be used in cloning to screen E. coli colonies for positive clones harboring plasmids containing an insert of interest. Separation of the amplification products from cHDA reactions of positive clones by gel electrophoresis allows detection of a specific target fragment and higher molecular weight bands that represent concatemers of the plasmid. The ability to simultaneously amplify the plasmid and identify plasmids of interest eliminates an additional screening step, such as restriction enzyme digestion of each purified plasmid.

(c) Since helicases can enzymatically denature DNA, the initial heat denaturation step required by the TempliPhi™ system can be omitted (GE Healthcare, formerly Amersham Biosciences, Piscataway, N.J.). Thus, the entire reaction can be performed at a single temperature, which further reduces the reaction steps.

(d) Amplification by cHDA may increase input nucleic acid levels by 10⁶.

(e) The amplification product from the cHDA system can be easily handled, digested, and cloned.

(f) Because specific primers are used, contaminating DNAs in the sample do not affect amplification. More importantly, a specific fragment defined by the primers is also amplified in addition to the whole plasmid amplification. Therefore, a single cHDA reaction can serve two purposes: screening for positive clones that display the specific target fragment and generating enough DNA for subsequent sequencing or cloning steps.

(g) Amplification of a broad range of sizes of DNA is possible by cHDA. For example, “long” cDNA” defined as any continuous sequence that is equal to or greater than 2 kb can be amplified. As shown in FIGS. 2 and 7, long multimers of plasmids greater than 40 kb are observed by gel electrophoresis and additional products that were too large to migrate into the agarose matrix were detected in the wells.

cHDA can also amplify short cDNA having a sequence length of 50 and 300 nucleotides.

Uses of c-HDA

Amplification of cDNA molecules by cHDA can be used as a tool in molecular biology applications and diagnostics, for example, when coupled with other technologies such as padlock probes [Nilsson et al., Science 265:2085-2088 (1994)]. Alternatively, small cDNA can be tagged to a molecule, such as an antibody to detect the binding of an antibody to an antigen using DNA amplification so as to increase immunodetection. [Schweitzer et al., Proc. Natl. Acad. Sci. USA 97:10113-10119 (2000)]. In immuno-HDA, a unique DNA sequence tag is associated with a specific antibody using streptavidin-biotin interactions, which is then detected by HDA of the DNA tag [Sano et al., Science 258:120-122(1992)].

cHDA may be used in disease diagnostics. For example, cHDA may be used to detect bacterial pathogens containing plasmids, such as Bacillus anthracis, [Tinsley et al., J. Bacteriology 186:2717-2723 (2004)]. In addition, cHDA may be used to amplify circular mitochondria DNA for the purpose of disease diagnosis and genome evolution studies.

The cHDA method can also be used to amplify large cDNAs, such as a plasmid DNA, from purified plasmid DNA or from a colony of bacterial cells. In the following examples, we disclose a helicase-based in vitro DNA amplification method that can be used to amplify circular nucleic acid molecules. More specifically, we describe a helicase-mediated plasmid amplification method that depends on a helicase and uses two specific primers.

Present embodiments of the invention are further illustrated by the following Examples. These Examples are provided to aid in the understanding of embodiments of the invention and are not construed as a limitation thereof.

The references cited above and below and provisional application 60/556,297 are herein incorporated by reference.

### Examples

### Example I

### Amplification of Purified cDNA Molecules Using cHDA

Experiment A: Methods to Perform cHDA

A pCR2.1 (Invitrogen, Carlsbad, Calif.) derivative plasmid, pREP, containing the E. coli rep helicase (GenBank accession number U00096) was used as a target template. Oligonucleotides S1224 and S1233 (New England Biolabs, Inc., Beverly, Mass.) that anneal to regions flanking the rep insert were used as primers. A 50 μl reaction was set up by mixing 5 μl of 10× cHDA Buffer (350 mM tris-acetate, pH 7.5, 110 mM Mg-acetate, 50 mM DTT), 10 ng pREP, 20 pmole S1224, 20 pmole S1233, 50 nmole dNTP, 500 nmole dTTP, 200 ng T7 gp 4B protein, 6 μg T7 gp2.5 protein, and 1 unit of T7 Sequenase (USB, Cleveland, Ohio). The reaction was incubated at 25° C. overnight and 5 μl of the reaction product was analyzed by separation through a 1% agarose gel containing ethidium bromide (FIG. 2A). A DNA fragment of approximately 2.3 kb size was observed (FIG. 2A, lane 1) and is consisted with the predicted size of the target sequence. In addition, higher molecular weight DNA bands corresponding to multiple repeats of plasmid DNA plus the insert in the form of concatemers were also observed (FIG. 2A, lane 1). The 2.3 kb amplification products were later sequenced and the sequencing results confirmed that the product was derived from the Rep gene.

Experiment B: Methods to Perform cHDA

A pCR2.1 (Invitrogen, Carlsbad, Calif.) derivative plasmid, pJK2, containing the E. coli rep helicase (GenBank accession number U00096) was used as a target template. Oligonucleotides S1263 and S1271 (NEB) that anneal to regions flanking the rep insert were used as primers. A 50 μl reaction was set up by mixing 5 μl of 10× cHDA Buffer (350 mM tris-acetate, pH 7.5, 110 mM Mg-acetate, 50 mM DTT), 15 ng pJK2, 20 pmole S1263, 20 pmole S1271, 50 nmole dNTP, 500 nmole dTTP, 600 ng T7 gp 4B protein, 6 μg T7 gp2.5 protein, and 2 units of T7 Sequenase (USB, Cleveland, Ohio). The reaction was incubated at 25° C. for 6 hours and 13 μl of the reaction product was analyzed by separation through a 1% agarose gel containing ethidium bromide.

T7gp4B helicase and T7 gp 2.5 SSB protein can be obtained by cloning using the DNA sequences for these proteins provided in GenBank (GenBank Accession Number V01146) using standard techniques (Molecular Cloning: A Laboratory Manual—www.MolecularCloning.com).

### Example III

### Optimization of cHDA Reaction Conditions

A. Determination of Specific Reagents Required for cHDA

A complete 50 μl reaction was set up as a control by mixing 5 μl of 10× cHDA Buffer (350 mM tris-acetate, pH 7.5, 110 mM Mg-acetate, 50 mM DTT), 10 ng pREP, 20 pmole S1224, 20 pmole S1233, 50 nmole dNTP, 500 nmole dTTP, 200 ng T7 gp 4B protein, 6 μg T7 gp2.5 protein, and 1 unit of T7 Sequenase (USB, Cleveland, Ohio). The requirement of each T7 protein was investigated. When one of the three T7 proteins was excluded from the reaction, no amplification product (FIG. 2A, lanes 2, 3, and 4) was observed and indicates that all three proteins are required for the cHDA reaction to proceed. Similarly, when both the helicase and SSB proteins were excluded, no amplification was detected (FIG. 2A, lane 5). Substitution of the T7 gp2.5 SSB protein with an equal amount of the T4 SSB did not support amplification (FIG. 2A, lane 6), indicating that a close coordination among the three T7 proteins is crucial for cHDA amplification.

B. Analyzing the Effects of an Initial Denaturation Step on cHDA

To investigate the effect of a prior denaturation step on cHDA reactions, two parallel reactions were conducted. One cHDA reaction was incubated at 25° C. for the entire reaction and, in the second reaction, plasmid templates and primers were first denatured at 95° C. for 3 minutes, then mixed with other cHDA components at 25° C. As shown in FIG. 2B, the amplification yield was slightly higher in the 2-step reaction than the one-step reaction.

C. Investigating the Primer Requirement for cHDA

To test whether both primers were needed for amplification, cHDA reactions were conducted with only one primer or with both primers using the pREP plasmid as the template. Parallel cHDA reactions were performed as described in part A of Example II. A positive control reaction contained both primers and two experimental reactions had only one primer, in which case water was substituted for the missing primer (FIG. 2C). Reactions were resolved by gel electrophoresis through a 1% agarose gel.

D. Optimization of cHDA Buffer Conditions

To determine optimum conditions for performing cHDA, various reagents were tested in cHDA at a range of concentrations. Standard cHDA reactions were performed, as described in Example II, Experiment B and Triton X-100, ZnCl₂, PEG-8000 or potassium glutamate were tested in a range of concentrations. Results of reactions containing the additional reagent were compared to a positive control reaction that did not contain any additional reagents. Reactions were resolved by gel electrophoresis through a 1% agarose gel (FIG. 3).

### Example IV

### Characterization of cHDA Products

A. Restriction Enzyme Digestion of cHDA Products

Restriction enzyme digestions of the cHDA amplification products were performed to further verify that tandem repeats of the plasmid containing the target fragment were produced. The restriction enzymes Acc65I, SacI, and XhoI (New England Biolabs, Inc., Beverly, Mass.) have a unique site within the pREP plasmid (FIG. 4A). The product from a cHDA reaction performed using the pREP plasmid was used in a series of restriction enzyme digests. After performing cHDA reactions, Acc65I, SacI and/or XhoI was directly added to the reaction and incubated at 37° C. for 6 hours. Following enzymatic digestion, the reactions were separated by gel electrophoresis through a 1% agarose gel. Results from the digests indicated that the large molecular weight amplification products are the plasmid in tandem repeats, forming a concatemer (FIG. 4B).

B. Analysis of Higher Molecular Weight cHDA Products

To analyze the higher molecular weight amplification products, a pulse-field gel electrophoresis was performed. DNA samples were separated through a 1% low melt agarose gel in a contour clamped homogenous electric field at 6 volts/cm. The gel was run with a switch time of 1.5 to 11 seconds at 14° C. for 16.5 hours. A ladder corresponding to multiple repeat units of 6-kb plasmid was observed (FIG. 4C).

C. Sequencing of cHDA Product

To test whether the reaction products can be used directly for sequencing with an ABI sequencer, a standard cHDA reaction, as described for part A of Example II, was performed using the pREP plasmid. Following the completion of the cHDA reaction, 1 μl of the cHDA reaction products was directly used for sequencing with S1224 (FIG. 5A) or a Rep-specific primer (FIG. 5B). Sequencing reactions were performed using an ABI sequencer following the manufacturer's recommendations. The sequencing reactions offered confident readings up to 600 bp.

### Example V

### Amplification of Crude Circular DNA

A. Methods for Performing cHDA Using Crude Circular DNA

Crude cDNA, in the form of plasmids, was obtained by heating colony cells resuspended in resuspension buffer (20 mM tris-acetate, pH 7.5) without further purification for use in cHDA reactions. Colony cells were suspended in 20 μl resuspension buffer and 5 μl of the resuspension buffer containing 1-5 μl of the colony suspension was incubated at 95° C. for 3 minutes, then cooled to 25° C. A 45 μl mix containing 1 unit of Sequenase (USB, Cleveland, Ohio), 7 μg T7 gp2.5 SSB protein, 250 ng T7 gp4B helicase, 50 pmole dNTP, 500 nmole dTTP, 5 pmole S1224, 5 pmole S1233, and 5 μl 10× cHDA buffer (350 mM tris-acetate, pH7.5, 110 mM Mg acetate, 50 mM DTT) was added to each tube and incubated at 25° C. for 6 hrs. Products of the cHDA reactions were resolved by gel electrophoresis through a 1% agarose gel. As shown in FIG. 6, the amplification products from a colony included a 2.3 kb fragment and higher molecular weight molecules, which is consistent with the cHDA products of the purified pREP plasmid (FIG. 6).

B. Analysis of Products from Crude Circular DNA cHDA Reactions

The crude cDNA from resuspended colony cells without purification was used for cHDA, as described in part A of Example V. Following amplification, 1 ml of the cHDA product was directly used for sequencing using S1224 or rep-specific primers. Sequencing reactions were performed using an ABI sequencer following the manufacturer's recommendations. Results indicated that the reactions specifically amplified the target region defined by the primers.

### Example VI

### Amplification of a Circular DNA Containing a Large Insert

A. Methods for Performing cHDA Using a Large Template DNA

To determine whether cHDA was effective on a large cDNA, reactions were performed using a pCR2.1 plasmid derivative, pTOPO10k, which contained a 10-kb target insert as the template. The oligonucleotides S1224 and S1233 (New England Biolabs, Inc., Beverly, Mass.) that flank the insert were used as primers. A 50-μl reaction was set up by mixing 5 μl of 10× cHDA Buffer, 20 ng pTOPO10k, 20 pmole S1224, 20 pmole S1233, 50 nmole dNTP, 500 nmole dTTP, 200 ng T7 gp4B protein, 6 μg T7 gp2.5 SSB protein, and 1 unit of T7 Sequenase. After an overnight reaction, 5 μl of each reaction was separated through a 1% agarose gel and stained with ethidium bromide (FIG. 7A). A 10 kb DNA band and DNAs of larger size were detected on the gel, which corresponded to the insert size and predicted concatemers, respectively.

B. Analyzing cHDA Products from a Large Template DNA

DNA samples were resolved by pulse-field gel electrophoresis through a 1% low melt agarose in a contour clamped homogenous electric field at 6 volts/cm. The gel was run with a switch time of 1.5 to 11 seconds at 14° C. for 16.5 hours. Results indicated a ladder pattern of DNA fragments was observed (FIG. 7B). The amplification products from pTopo10k were cleaved into the insert and plasmid fragments by restriction enzyme digestion, and were also directly used in sequencing reactions.

### Example VII

### The Use of cHDA in Diagnostics

To identify and detect bacterial plasmids encoding genes for pathogenesis and/or antibiotic resistance, cHDA is used to directly analyze cells. Potential pathogens are concentrated in biological samples by, for example, centrifugation. The concentrated sample is then resuspended in a small volume of lysis solution that may include detergents, and/or enzymes to lyse the cell wall and membrane of bacteria or bacterial spores. Details of bacterial spore lysis have been previously described [Ryu et al., Miocrobiol. Immunol. 47:693-699 (2003)]. Heat (90°-100° C.) may also be used to help lyse cells. A sample of the lysed cells (10 μl) is combined with 40 μl of the cHDA mixture, including the T7 Sequenase, T7 gp2.5 SSB protein, T7 gp4B helicase, dNTP, 500 nmole dTTP, primers specific to the target sequence in the plasmid, and cHDA buffer (detailed in Example III). Following the cHDA reaction, amplified products are analyzed by various methods, including agarose gel electrophoresis, immuno-chromatographic strips [Matsubara and Kure, Human Mutation 22:166-172 (2003)], or real time detection via fluorescence signals. To further analyze the pathogenic plasmids, the amplified products are digested with restriction enzymes to generate footprints of the plasmid for further study.

### Example VIII

### A Kit for Plasmid DNA Amplification

The methods disclosed in embodiments of the invention may be incorporated into a kit. For example, a kit for amplifying plasmid DNA may be commercialized in the following novel composition. A cHDA plasmid amplification kit may be composed of a colony suspension buffer, a cHDA reaction mixture containing reaction buffer, a DNA polymerase, four dNTPs (dATP, dGTP, dCTP, dTTP), and a helicase preparation, and accessory proteins, if any. The kit also includes a manual of how to perform plasmid DNA amplification according to Examples II and III. The kit may also include a control plasmid and a pair of primers specific for the control plasmid.

What is claimed is:

1. A method for amplifying a target DNA sequence in a circular DNA (cDNA), comprising:
(a) providing the cDNA template;
(b) adding to the cDNA template:
(i) a first sequence-specific DNA primer that hybridizes to one strand of the cDNA;
(ii) a second sequence-specific DNA primer that is substantially identical to a portion of the one strand of the cDNA; and
(iii) a helicase preparation, a DNA polymerase and dNTPs;
(c) synthesizing primer extension products to produce:
(i) a plurality of copies of the target DNA sequence defined by the first and second primers; and
(ii) a plurality of copies of a concatamer derived from the cDNA including the target; and
(d) amplifying the target DNA sequence.

2. A method according to claim 1, wherein the target DNA sequence is the entire cDNA.

3. A method according to claim 1, wherein the target DNA sequence is a defined sequence within the cDNA bordered by the first and second primers.

4. A method according to claim 3, wherein the first specific sequence on the cDNA recognized by the first primer does not overlap with the second specific sequence recognized by the second primer.

5. A method according to claim 3, wherein the first specific sequence on the cDNA recognized by the first primer overlaps with the second specific sequence recognized by the second primer.

6. A method according to claim 1, wherein the cDNA is a duplex or is single-stranded or is part double-stranded with a residual part being single-stranded.

7. A method according to claim 1, wherein the DNA polymerase is selected from the group consisting of a T7 bacteriophage DNA polymerase, a T7-like polymerase and an exonuclease-deficient variant thereof.

8. A method according to claim 1, wherein the helicase preparation comprises a processive helicase.

9. A method according to claim 8, wherein the processive helicase is a hexameric-replicative helicase.

10. A method according to claim 9, wherein the processive helicase is obtained from T7 bacteriophage or T7-like bacteriophage.

11. A method according to claim 9, wherein the helicase preparation comprises a single-strand binding protein from a T7 bacteriophage or T7-like bacteriophage.

12. A method according to claim 1, wherein the helicase preparation comprises nucleotides.

13. A method according to claim 1, wherein the cDNA is a plasmid DNA.

14. A method according to claim 1, wherein the cDNA has a size in the range of 50 bp-500 kb.

15. A method according to claim 1, wherein the amplification in step (b) is isothermal.

16. A method according to claim 15, wherein the isothermal temperature is approximately 25° C.

17. A method according to claim 1, wherein the cDNA is extrachromosomal DNA for detecting a pathogen.

18. A method for amplifying a target DNA sequence in a cDNA, comprising:
(a) providing the cDNA;
(b) contacting the cDNA with a first sequence specific oligonucleotide primer that hybridizes to one strand of the cDNA and a second sequence specific oligonucleotide primer that is substantially identical to a portion the one strand of the cDNA, a helicase preparation, a DNA polymerase, and dNTPs;
(c) replicating the cDNA under conditions whereby the first primer is extended around the circle repeatedly to generate a single strand linear concatemer, and whereby the second primer hybridizes to multiple sites on a complementary single-stranded DNA which generate extension products for continuing synthesis of the target DNA sequence and the cDNA; and
(d) amplifying the target cDNA.

19. A method for amplifying a target DNA sequence in a cDNA, comprising:
(a) annealing a first sequence specific oligonucleotide primer to a first DNA sequence adjacent to or within a target DNA sequence in a cDNA in the presence of a polymerase and a helicase preparation to synthesize by primer extension, a displaced single stranded DNA containing a plurality of copies of the target DNA sequence;
(b) annealing a second specific oligonucleotide primer to the displaced strand of DNA at specific sites for synthesizing by primer extension, a plurality of complementary single stranded DNA concatamers, each concatamer including the target DNA sequence and each concatemer forming a substrate for multiple rounds of displacement synthesis from the first or second primer; and
(c) amplifying the target DNA sequence.

20. An amplification kit comprising: a helicase preparation containing one or more helicases, a DNA polymerase and instructions for performing helicase-dependent amplification of circular nucleic acids according to claim 1.

21. An amplification kit according to claim 20, wherein the helicase preparation comprises: T7 gene 4B helicase, T7 gene 2.5 SSB protein, dTTP or ATP, T7 Sequenase.

22. An amplification kit according to claim 21, wherein the helicase preparation further comprising one or more cofactors including an accessory protein, a set of four deoxynucleotides and optionally a reaction buffer.


SEQUENCE LISTING (6 sequences)

SEQ ID NO: 1 - DNA, 34 bp, unknown
  primer 4B51
accctttcatatgacttacaacgtgtggaacttc

SEQ ID NO: 2 - DNA, 37 bp, unknown
  primer 4B31
ggaaatgctcttccgcagaagtcagtgtcgttggacc

SEQ ID NO: 3 - DNA, 107 bp, unknown
  primer 2551
accctttcatatggctaagaagattttcacctctgcgctgggtaccgctgaaccttacgcttacatcgccaagccggactacggcaacgaggagcgtggctttggga

SEQ ID NO: 4 - DNA, 37 bp, unknown
  primer 2531
ggaaatgctcttccgcagaagtctccgtcttcgtctg

SEQ ID NO: 5 - DNA, 64 bp, unknown
  sequencing trace data using primer S1224
ttgcttaaagagttgaccgaggggctgattgaagatgacaaagttctcctgcaacaactgattt

SEQ ID NO: 6 - DNA, 64 bp, unknown
  region of sequencing trace data using a Rep specific primer
tgatctcaaaacaccgtcccaggcggcagcaagtgcgattggcgagcgggaccgtatttttgcc